(19) United States
(12) Patent Application Publication
(10) Pub. No.: US 2024/0226512 A1
(43) Pub. Date: Jul. 11, 2024

(54) TAPERED BALLOON

(71) Applicant: BOSTON SCIENTIFIC SCIMED, INC., MAPLE GROVE, MN (US)

(72) Inventors: Daryl Lee Martin, Plymouth, MN (US); Jeffrey S. Lindquist, Maple Grove, MN (US); Joel M. WasDyke, Independence, MN (US)

(73) Assignee: BOSTON SCIENTIFIC SCIMED, INC., MAPLE GROVE, MN (US)

(21) Appl. No.: 18/403,261

(22) Filed: Jan. 3, 2024

Related U.S. Application Data
(60) Provisional application No. 63/437,439, filed on Jan. 6, 2023.

(51) Int. Cl. A61M 25/10

(52) U.S. Cl. CPC A61M 25/1029; A61M 2207/00

(57) ABSTRACT

Balloon catheters having a tapered balloon design and methods for manufacturing tapered balloons. An illustrative method for forming a tapered balloon for use in a medical device may comprise forming a balloon tube, the balloon tube may be formed from a first material and a second material different from the first material, positioning the balloon tube within a balloon mold, and blow molding the tapered balloon tube within the balloon mold to form a balloon.

### CROSS REFERENCE TO RELATED APPLICATIONS

This application claims the benefit of U.S. Provisional Patent Application Ser. No. 63/437,439 filed Jan. 6, 2023, the disclosure of which is incorporated herein by reference.

### TECHNICAL FIELD

The disclosure pertains to balloon catheters. More particularly, the disclosure is directed to balloon catheters having a tapered balloon design and methods for manufacturing tapered balloons.

### BACKGROUND

Balloon catheters may be used to widen or enlarge passages in the body, such as, but not limited to, within the vasculature. In some instances, it may be desirable to treat long vessel sections with a single balloon. These long vessel sections may reduce in diameter along a length thereof. Thus, it may be desirable to provide a balloon which decreases in diameter along a length thereof. Of the known medical devices, systems, and methods, each has certain advantages and disadvantages. There is an ongoing need to provide alternative medical devices and systems, including devices and systems for treating variable diameter passages in the body.

### SUMMARY

The disclosure is directed to several alternative designs, materials and methods of manufacturing medical device structures and assemblies.

In a first example, a method for forming a tapered balloon for use in a medical device may comprise forming a balloon tube, the balloon tube formed from a first material and a second material different from the first material, positioning the balloon tube within a balloon mold, and blow molding the tapered balloon tube within the balloon mold to form a balloon.

Alternatively or additionally to any of the examples above, in another example, the first material may be more compliant than the second material.

Alternatively or additionally to any of the examples above, in another example, the balloon tube may decrease in outer diameter from a proximal end to a distal end.

Alternatively or additionally to any of the examples above, in another example, an outer diameter of the balloon tube may be uniform from a proximal end to a distal end.

Alternatively or additionally to any of the examples above, in another example, the balloon tube may comprise a proximal end region comprising the first material and a distal end region comprising the second material.

Alternatively or additionally to any of the examples above, in another example, the proximal end region may be more compliant than the distal end region.

Alternatively or additionally to any of the examples above, in another example, the balloon tube may further comprise a transition region between the proximal end region and the distal end region.

Alternatively or additionally to any of the examples above, in another example, the transition region may comprise a gradient transition from the first material to the second material.

Alternatively or additionally to any of the examples above, in another example, the balloon tube may comprise an inner layer comprising the first material and an outer layer comprising the second material.

Alternatively or additionally to any of the examples above, in another example, a wall thickness of the inner layer may decrease from a proximal end to a distal end of the balloon tube.

Alternatively or additionally to any of the examples above, in another example, a wall thickness of the outer layer may increase from a proximal end to a distal end of the balloon tube.

Alternatively or additionally to any of the examples above, in another example, a ratio of a wall thickness of the inner layer to a wall thickness of the outer layer may be varied along a length of the balloon tube.

Alternatively or additionally to any of the examples above, in another example, a hoop ratio of the balloon mold and the balloon tube may be consistent along a length of a body portion of the formed balloon.

Alternatively or additionally to any of the examples above, in another example, the balloon mold may comprise a body portion that reduces in diameter in a distal direction.

Alternatively or additionally to any of the examples above, in another example, the balloon mold may comprise a body portion that has generally constant diameter.

In another example, a method for forming a tapered balloon for use in a medical device may comprise forming a tapered balloon tube, the tapered balloon tube decreasing in outer diameter from a proximal end to a distal end, positioning the tapered balloon tube within a balloon mold, the balloon mold having a body portion that reduces in diameter in a distal direction, and blow molding the tapered balloon tube within the balloon mold to form a balloon.

Alternatively or additionally to any of the examples above, in another example, positioning the tapered balloon tube within the balloon mold may comprise aligning a proximal diameter transition point of the balloon tube with a proximal end of the body portion of the balloon mold.

Alternatively or additionally to any of the examples above, in another example, a hoop ratio of the balloon mold and the balloon tube may be consistent along a length of a body portion of the formed balloon.

Alternatively or additionally to any of the examples above, in another example, the tapered balloon tube may be formed from a single material.

In another example, a method for forming a tapered balloon for use in a medical device, may comprise forming a balloon tube, the balloon tube formed from a first material and a second material different from the first material, positioning the balloon tube within a balloon mold, and blow molding the tapered balloon tube within the balloon mold to form a balloon.

Alternatively or additionally to any of the examples above, in another example, the first material may be more compliant than the second material.

Alternatively or additionally to any of the examples above, in another example, the balloon tube may decrease in outer diameter from a proximal end to a distal end.

Alternatively or additionally to any of the examples above, in another example, an outer diameter of the balloon tube may be uniform from a proximal end to a distal end.

Alternatively or additionally to any of the examples above, in another example, the balloon tube may comprise a proximal end region comprising the first material and a distal end region comprising the second material.

Alternatively or additionally to any of the examples above, in another example, the proximal end region may be more compliant than the distal end region.

Alternatively or additionally to any of the examples above, in another example, the balloon tube may further comprise a transition region between the proximal end region and the distal end region.

Alternatively or additionally to any of the examples above, in another example, the transition region may comprise a gradient transition from the first material to the second material.

Alternatively or additionally to any of the examples above, in another example, the balloon tube may comprise an inner layer comprising the first material and an outer layer comprising the second material.

Alternatively or additionally to any of the examples above, in another example, a wall thickness of the inner layer may decrease from a proximal end to a distal end of the balloon tube.

Alternatively or additionally to any of the examples above, in another example, a wall thickness of the outer layer may increase from a proximal end to a distal end of the balloon tube.

Alternatively or additionally to any of the examples above, in another example, a hoop ratio of the balloon mold and the balloon tube may be consistent along a length of a body portion of the formed balloon.

In another example, a method for forming a tapered balloon for use in a medical device may comprise forming a tapered balloon tube, the tapered balloon tube decreasing in outer diameter from a proximal end to a distal end and formed from a first material and a second material, wherein the first material is more compliant that the second material, positioning the balloon tube within a balloon mold, and blow molding the tapered balloon tube within the balloon mold to form a balloon.

Alternatively or additionally to any of the examples above, in another example, the balloon tube may comprise a proximal end region comprising the first material and a distal end region comprising the second material.

Alternatively or additionally to any of the examples above, in another example, the balloon tube may further comprise a transition region between the proximal end region and the distal end region.

Alternatively or additionally to any of the examples above, in another example, the balloon tube may comprise an inner layer comprising the first material and an outer layer comprising the second material.

Alternatively or additionally to any of the examples above, in another example, a ratio of a wall thickness of the inner layer to a wall thickness of the outer layer may be varied along a length of the balloon tube.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a side view of an illustrative balloon catheter;

FIG. 2 is a schematic cross-section of an illustrative balloon tube that may be used to form a tapered balloon;

FIG. 3 is an illustrative flowchart of a method for forming a tapered balloon;

FIG. 4 is a schematic cross-section of another illustrative balloon tube that may be used to form a tapered balloon;

FIG. 5 is an illustrative flowchart of another method for forming a tapered balloon;

FIG. 6 is a schematic cross-section of another illustrative balloon tube that may be used to form a tapered balloon; and

FIG. 7 is an illustrative flowchart of another method for forming a tapered balloon.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. Further, as used herein, the terms “about,” “approximately” and “substantially” indicate a range of values within +/−10% of a stated or implied value. Additionally, terms that indicate the geometric shape of a component/surface refer to exact and approximate shapes.

As used herein, a “body portion” of a balloon refers to the generally central portion of the balloon between the cone portions of the balloon. The body portion is typically the portion of the balloon with the largest width or diameter when the balloon is fully inflated. A “cone portion” of a balloon refers to a portion of the balloon that has a variable (e.g., tapered) width or diameter extending between the body portion and a waist portion. The “waist portion” of a balloon refers to the portion of the balloon that contacts a portion of catheter shaft on the proximal and distal ends of the balloon. The waist portion is typically the portion of the balloon with the smallest width or diameter. The waist portion, for example, can have an inner diameter that is substantially equal to the outer diameter of the portion of the catheter shaft to which it contacts. As used herein, “tapered” is used to describe a gradually increasing or decreasing diameter.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Balloon catheters may be used to widen or enlarge passages in the body, such as, but not limited to, within the vasculature. In some instances, it may be desirable to treat long vessel sections with a single, long balloon. These long vessel sections may reduce in diameter along a length thereof. Thus, it may be desirable to provide a balloon which decreases in diameter along a length thereof. Existing tapered balloons have a varied balloon wall along the length. This may result in inconsistent radial growth of the balloon when the long-tapered balloon is inflated. For example, the balloon may expand to a greater extent on one end relative to the other. Further, the thicker distal wall may result in less-than optimal distal deliverability. The present disclosure is directed towards tapered balloons which have a greater ability to control the radial grown of the balloon diameter.

A typical method for forming medical device balloons, such as the tapered balloons described herein, may include molding the balloon from a balloon tube, such as, but not limited to, an extruded balloon tube, in a mold during a blow molding process. During the blow molding process, the mold is heated to elevate the temperature of the polymeric material (e.g., thermoplastic material) of the balloon tube in order to soften the polymeric material. While the balloon tube is at an elevated temperature, the interior of the balloon tube is pressurized to expand the softened polymeric material within the cavity of the mold such that the polymeric material conforms to the shape of the cavity to form the inflatable balloon. The cavity within the balloon mold, defined by an inner surface of the balloon mold, may be configured to be the size and shape of the desired size and shape of the balloon to be formed from the balloon tube within the balloon mold during the blow molding process. During a subsequent manufacturing process, the formed balloon may be bonded to a catheter shaft to form the balloon catheter (e.g., the balloon waists are bonded to the catheter shaft after the balloon is blow molded).

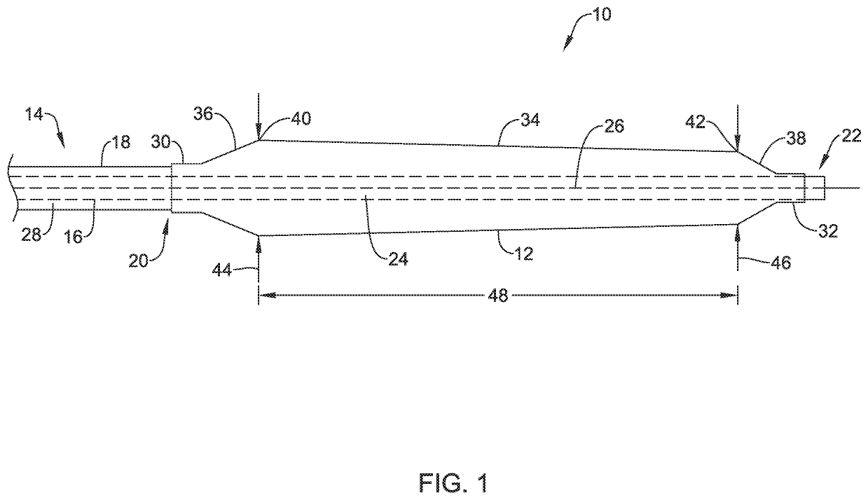

FIG. 1 is a side view of an illustrative balloon catheter 10. The balloon catheter 10 may be used to treat lesions in a blood vessel. However, the balloon catheter 10 may be used in other anatomy in addition to the vasculature, as desired. The balloon catheter 10 may include a balloon 12 coupled to an elongate shaft 14. In general, the catheter 10 may be advanced over a guidewire 26, through the vasculature, to a target area with the balloon 12 in a collapsed or deflated configuration. Once positioned at the target location in the vasculature, the balloon 12 can be inflated to exert a radially outward force on the vessel wall. The target area may be within any suitable peripheral or cardiac vessel lumen location.

The shaft 14 may be a catheter shaft, similar to typical catheter shafts. For example, the catheter shaft 14 may include an inner tubular member 16 and an outer tubular member 18, the inner tubular member 16 extending through at least a portion of the outer tubular member 18. The inner and outer tubular members 16, 18 may be manufactured from a number of different materials. For example, the inner and outer tubular members 16, 18 may be made of metals, metal alloys, polymers, metal-polymer composites or any other suitable materials.

The outer tubular member 18 may extend proximally from a distal end region 20 to the proximal end configured to remain outside of a patient's body. The inner tubular member 16 may extend proximally from a distal end region 22 to a proximal end configured to remain outside of a patient's body. Although not shown, the proximal ends of the inner and/or outer tubular members 16, 18 may include a hub attached thereto for connecting other treatment devices or providing a port for facilitating other treatments. It is contemplated that the stiffness of the inner and/or outer tubular members 16, 18 may be modified to form a catheter 10 for use in various vessel diameters and various locations within the vascular tree.

The inner and outer tubular members 16, 18 may be arranged in any appropriate way. For example, in some embodiments, the inner tubular member 16 can be disposed coaxially within the outer tubular member 18. According to these embodiments, the inner and outer tubular members 16, 18 may or may not be secured to one another along the general longitudinal axis of the catheter shaft 14. Alternatively, the inner tubular member 16 may follow the inner wall or otherwise be disposed adjacent the inner wall of the outer tubular member 18. In other embodiments, the inner and outer tubular members 16, 18 may be arranged in another desired fashion. In some embodiments, the inner tubular member 16 and/or outer tubular member 18 may be torqueable to facilitate rotation of the device 10. The inner tubular member 16 and/or outer tubular member 18 may include an embedded reinforcing member, such as, but not limited to, an embedded coil or braided member.

The inner tubular member 16 may include an inner lumen 24. In at least some embodiments, the inner lumen 24 is a guidewire lumen for receiving the guidewire 26 therethrough. Accordingly, the catheter 10 can be advanced over the guidewire 26 to the desired location. The guidewire lumen 24 may extend along essentially the entire length of the catheter shaft 14 such that catheter 10 resembles traditional “over-the-wire” catheters. Alternatively, the guidewire lumen 24 may extend along only a portion of the catheter shaft 14 such that the catheter 10 resembles “single-operator-exchange” or “rapid-exchange” catheters.

The catheter shaft 14 may also include an inflation lumen 28 that may be used, for example, to transport inflation media to and from the balloon 12 to selectively inflate and/or deflate the balloon 12. The location and position of the inflation lumen 28 may vary, depending on the configuration of the inner and outer tubular members 16, 18. For example, when the outer tubular member 18 surrounds the inner tubular member 16, the inflation lumen 28 may be defined within the space between the outer tubular member 18 and the inner tubular member 16. In embodiments in which the outer tubular member 18 is disposed alongside the inner tubular member 16, then the inflation lumen 28 may be the lumen of the outer tubular member 18.

The balloon 12 may be coupled to the catheter shaft 14 in any of a number of suitable ways. For example, the balloon 12 may be adhesively or thermally bonded to the catheter shaft 14. In some embodiments, a proximal waist 30 of the balloon 12 may be bonded to the catheter shaft 14, for example, bonded to the distal end region 20 of the outer tubular member 18, and a distal waist 32 of the balloon 12 may be bonded to the catheter shaft 14, for example, bonded to the distal end region 22 of the inner tubular member 16. The exact bonding positions, however, may vary.

The balloon 12 may include a central body portion 34, a proximal cone 36, and a distal cone 38. The proximal cone 36 may extend between the proximal end 40 of the body portion 34 and the proximal waist portion 30. When the balloon 12 is the expanded configuration, the proximal cone 36 may taper or reduce in diameter in the proximal direction. The distal cone 38 may extend between the distal end 42 of the body portion 34 and the distal waist portion 32. When the balloon 12 is the expanded configuration, the distal cone 38 may taper or reduce in diameter in the distal direction.

In the expanded configuration (as shown in FIG. 1), the body portion 34 of the balloon 12 may taper or reduce in diameter from the proximal end 40 to the distal end 42 thereof. For example, the body portion 34 may have a first outer diameter 44 adjacent to the proximal end 40 and a second outer diameter 46 adjacent to the distal end 42. The second outer diameter 46 may be less than the first outer diameter 44. The outer diameter of the body portion 34 may reduce in a sloped or tapered manner such that the change from the first outer diameter 44 to the second outer diameter 46 is a constant gradual change. In some examples, the first outer diameter 44 may be in the range of about 3 millimeters (mm) to about 4 mm, or about 3.5 mm, and the second outer diameter 46 may be in the range of about 2 mm to about 3 mm, or about 2.5 mm. For example, the body portion 34 may have a taper (or a reduction in outer diameter) of about 1 mm from the proximal end 40 to the distal end 42 thereof. The inside diameter of the body portion 34 may taper in a similar manner such that a wall thickness of the body portion 34 remains substantially constant along a length 48 of the body portion 34, although this is not required. It is contemplated that the length 48 of the body portion 34 may be selected for a desired treatment location. In some cases, the length 48 may be about 300 mm. In some examples, the length 48 may be greater than 300 mm. In other examples, the length 48 may be less than 300 mm.

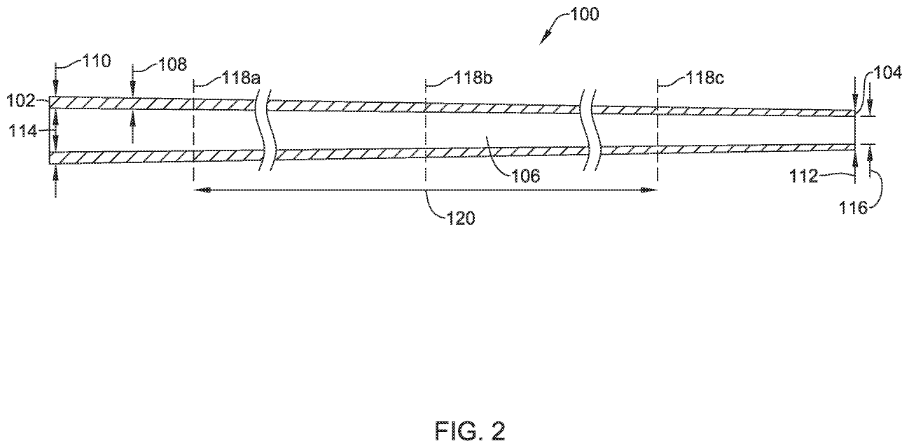

FIG. 2 is a schematic cross-section of an illustrative balloon tube 100 that may be used to form a tapered balloon, such as, but not limited to the balloon 12 described herein. The balloon tube 100 may extend from a proximal end 102 to a distal end 104. A lumen, or inner diameter, 106 may extend from the proximal end 102 to the distal end 104. Generally, the balloon tube 100 may be a tapered tube or a bumped tube. For example, the balloon tube 100 may reduce in outer diameter from a first outer diameter 110 adjacent the proximal end 102 to a second, smaller, outer diameter 112 adjacent the distal end 104 thereof. Additionally, or alternatively, the balloon tube 100 may reduce in inner diameter from a first inner diameter 114 adjacent the proximal end 102 to a second, smaller inner diameter 116 adjacent the distal end 104 thereof. In the illustrated embodiment, both the outer and the inner diameters gradually reduce towards the distal end 104. In some examples, the outer diameter of the balloon tube 100 may have a similar slope or diameter change rate as the inner diameter of the balloon tube 100 so that a wall thickness 108 of the balloon tube 100 remains substantially constant along a length of the balloon tube 100. In other examples, the outer diameter the outer diameter of the balloon tube 100 may have a differing slope or diameter change rate from the inner diameter of the balloon tube 100 so that a wall thickness 108 of the balloon tube 100 decreases from the proximal end 102 to the distal end 104 thereof. In some instances, the difference between the first outer diameter 110 and the second outer diameter 112 may be greater than the difference between the first inner diameter 114 and the second inner diameter 116. The reverse configuration in which the difference between the first inner diameter and the second inner diameter is greater than the difference between the first outer diameter and the second outer diameter is also contemplated.

During the blow molding process, an intermediate portion 120 of the balloon tube 100 may form the body portion of the balloon. The intermediate portion 120 may extend from a proximal diameter transition point 118a to a distal diameter point 118c. To form a balloon having a body portion length of about 300 mm, the intermediate portion 120 may have a length in the range of about 4.6 inches (116.8 mm) to about 4.8 inches (121.9 mm), or about 4.7 inches (119.4 mm). However, the length of the intermediate portion 120 may be selected to form the desired balloon length. For example, a shorter balloon body portion may be formed from a shorter balloon tube and a longer balloon body portion may be formed from a longer balloon tube. At the proximal diameter transition point 118a, the outer diameter may be in the range of about 0.039 inches (0.99 mm) to about 0.043 inches (1.09 mm), or about 0.041 inches (1.04 mm) and the inner diameter may be in the range of about 0.023 inches (0.58 mm) to about 0.027 inches (0.69 mm), or about 0.025 inches (0.64 mm). At the distal diameter transition point 118c, the outer diameter may be in the range of about 0.031 inches (0.79 mm) to about 0.035 inches (0.89 mm), or about 0.033 inches (0.84 mm) and the inner diameter may be in the range of about 0.018 inches (0.46 mm) to about 0.022 inches (0.56 mm) or about 0.020 inches (0.51 mm). An intermediate outer diameter, for example, at or near the second diameter transition point 118b, may be in the range of about 0.035 inches (0.89 mm) to about 0.039 inches (0.99 mm) or about 0.037 inches (0.94 mm) and an intermediate inner diameter may be in the range of about 0.020 inches (0.51 mm) to about 0.024 inches (0.61 mm) or about 0.022 inches (0.56 mm). It is contemplated that the outer diameters, inners diameters, and/or a rate of change of the outer and/or inner diameters may be varied, as desired.

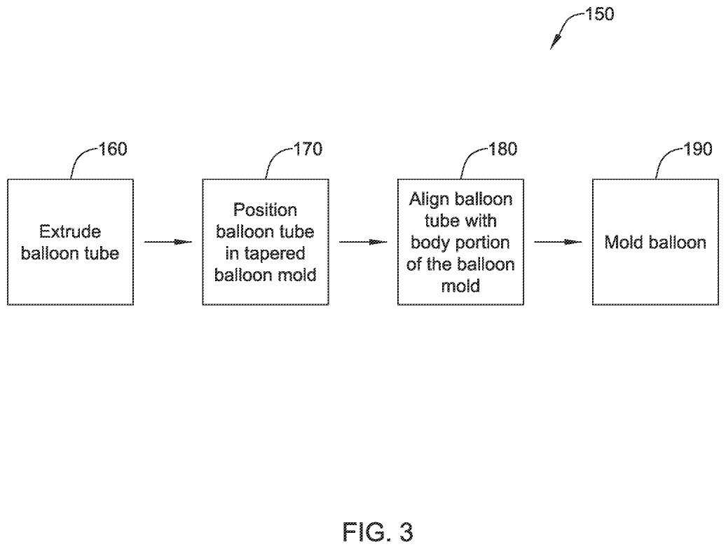

FIG. 3 is an illustrative flowchart of a method 150 for forming a tapered balloon using a tapered (or bumped) balloon tube, such as, but not limited to, balloon tube 100. To begin, the balloon tube 100 may be extruded or otherwise formed, as shown at block 160. This may be done using known extrusion techniques for forming a tapered or bumped tube. The balloon tube 100 may then be positioned or placed into a tapered balloon mold, as shown at block 170. In some examples, the balloon tube 100 may be pre-stretched prior to positioning the balloon tube 100 into the tapered balloon mold to increase a length of the balloon tube 100 and/or reduce a wall thickness of the balloon tube 100 prior to molding. The balloon mold may be formed to have the desired dimensions of the balloon in the expanded configuration. For example, the balloon mold may have a body portion which tapers or reduces in the distal direction. The balloon tube 100 may then be adjusted to align a portion of the balloon tube 100 with the body portion of the balloon mold, as shown at block 180. In some examples, this may include aligning a proximal diameter transition point (e.g., transition point 118a in FIG. 2) with the proximal end of the body portion of the mold and a distal diameter transition point (e.g., transition point 118c in FIG. 2) with the distal end of the body portion of the mold. The balloon may then be molded using, for example, blow molding, as shown at block 190.

During the blow molding process, the mold is heated to elevate the temperature of the polymeric material (e.g., thermoplastic material) of the balloon tube 100 in order to soften the polymeric material. While the balloon tube 100 is at an elevated temperature, the interior of the balloon tube 100 may be pressurized to expand the softened polymeric material within the cavity of the mold such that the polymeric material conforms to the shape of the cavity to form the inflatable balloon. The cavity within the balloon mold, defined by an inner surface of the balloon mold, may be configured to be the size and shape of the desired size and shape of the balloon to be formed from the balloon tube 100 within the balloon mold during the blow molding process. During a subsequent manufacturing process, the formed balloon may be bonded to a catheter shaft to form the balloon catheter (e.g., the balloon waists are bonded to the catheter shaft after the balloon is blow molded).

The resultant balloon may have a consistent hoop ratio (or ID hoop) along a length of the body portion. For example, the resultant balloon may have a hoop ratio that is +0.1 along a length thereof. Other tolerance ranges may be used as desired. As used herein, hoop ratio is calculated as follows:

$$hoop ratio = \frac{balloon mold inner diameter}{balloon tube inner diameter} \qquad Equation 1$$

As can understood from Equation 1, in order to maintain a consistent hoop ratio along the length of the body portion, the inner diameter of the balloon mold may be formed to have a slope which mirrors the slope of the inner diameter of the balloon tube.

It is contemplated that consistent hoop ratios in balloons may be important when the objective is to maintain radial distension along the length of an inflated balloon. For example, a consistent hoop ratio may result in a consistent hoop stress along the length of the body portion thus allowing the radial compliance (e.g., percentage of radial balloon growth upon inflation) to remain consistent along a length of the body portion. However, the use of a standard balloon tube (e.g., a balloon tube which maintains its dimensions along the length of the tube) in a tapered balloon mold may result in a balloon with a hoop ratio which was greater at the proximal end of the body portion than the distal end of the body portion. Further, a tapered balloon formed from a uniform diameter balloon tube may also have a thicker balloon wall at the distal end of the body portion of the balloon as compared to the proximal end of the body portion. However, in contrast, to aid in deliverability, the balloon wall should be thinner on the distal end of the body portion. The use of the tapered or bumped balloon tube to form a tapered balloon, as described with respect to FIGS. 2 and 3, may result in a balloon having a thinner balloon wall at the distal end body portion relative to a balloon formed with a uniform diameter balloon tube molded in a tapered mold. Thus, a balloon formed using a tapered or bumped balloon tube and a tapered balloon mold may have both a consistent hoop ratio along a length thereof, which may maintain radial compliance of the body portion, and improved deliverability.

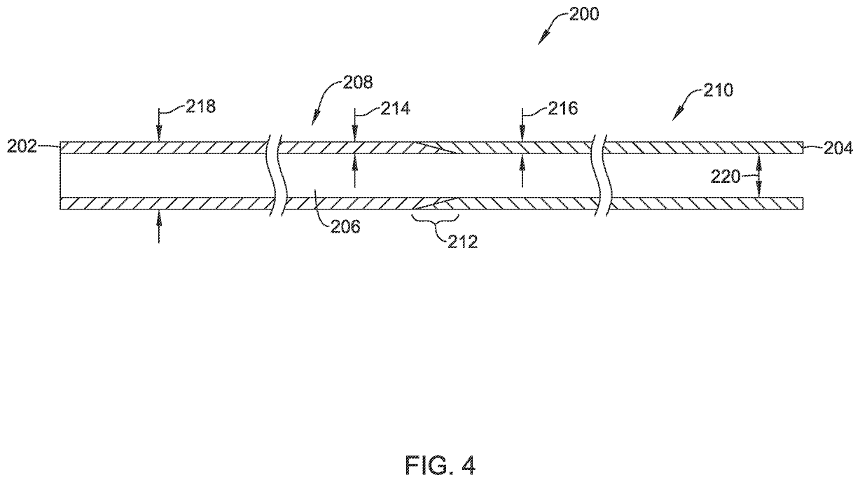

FIG. 4 is a schematic cross-section of another illustrative balloon tube 200 that may be used to form a tapered balloon, such as, but not limited to the balloon 12 described herein. The balloon tube 200 may extend from a proximal end 202 to a distal end 204. A lumen 206 may extend from the proximal end 202 to the distal end 204. Generally, the balloon tube 200 may include two or more different materials along a length thereof to vary the compliance characteristics of the balloon along the length thereof. Said differently, the material composition of the balloon tube 200 may vary from the proximal end 202 to the distal end 204. For example, the balloon tube 200 may include a proximal end region 208 formed from a first material and a distal end region 210 formed from a second material different from the first material. It is contemplated that the first material may be softer than the second material such that the proximal end region 208 expands to a greater diameter than the distal end region 210 under the same pressure to form a balloon having a body portion that reduces in diameter in the distal direction when inflated. Said differently, the proximal end region 208 may be more compliant than the distal end region 210. The proximal end region 208 may be formed from compliant or semi-compliant materials, such as, but not limited to polyurethanes, silicone, polyether block amides (such as, but not limited to, PEBAX®), higher durometer polyurethanes, etc. The distal end region 210 may be formed from semi-compliant or non-compliant materials such as, but not limited to, polyamides, polyesters, polyether block amides (such as, but not limited to, PEBAX® or VESTAMID®).

The balloon tube 200 may further include a transition region 212 between the proximal end region 208 and the distal end region 210. The transition region 212 may include a gradient transition from the first material to the second material. For example, a wall thickness 214 of the proximal end region 208 may decrease in the distal direction along the transition region 212 while the wall thickness 216 of the distal end region 210 may increase in the distal direction along the transition region 212. It is contemplated that the transition region 212 may be positioned anywhere along a length of the balloon tube 200. In some examples, the transition region 212 may be at the approximate middle of the balloon tube 200. In other examples, the transition region 212 may be positioned anywhere between the proximal end 202 and the distal end 204. It is contemplated that the location of the transition region 212 may be based on the desired profile of the inflated location. Further, if more than two materials are used, the balloon tube 200 may include more than one transition region 212. It is further contemplated that a length of the transition region 212 may vary based on the desired profile of the inflated balloon. It is contemplated that the transition region 212 may be very short (e.g., in the range of a few millimeters) to very long. For example, in some cases, the transition region 212 may extend into at least one of the resultant proximal or distal balloon cones. In other examples, the transition region 212 may extend along an entire length of the resultant balloon body portion. These are just some examples. The transition region 212 may have any length desired.

In the illustrated embodiments, the outer diameter 218 and the inner diameter 220 of the balloon tube 200 remain constant along a length of the balloon tube 200. However, this is not required. In some examples, the outer diameter 218 and/or the inner diameter 220 may decrease in the distal direction, if so desired, as described with respect to FIG. 2, so that the balloon tube 200 is tapered.

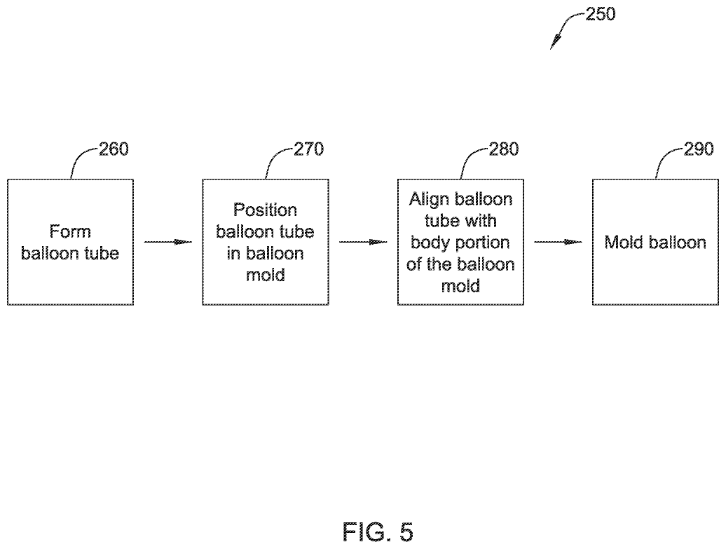

FIG. 5 is an illustrative flowchart of a method 250 for forming a tapered balloon using a multi-material tube, such as, but not limited to, balloon tube 200. To begin, the balloon tube 200 may be formed, as shown at block 260. In some embodiments, the balloon tube 200 may be extruded as a single monolithic structure with multiple layers. In other embodiments, the balloon tube 200 may be formed as separate components that are subsequently coupled at the transition region 212. The balloon tube 200 may then be placed into a balloon mold, as shown at block 270. In some examples, the balloon tube 200 may be pre-stretched prior to positioning the balloon tube 200 into the tapered balloon mold to increase a length of the balloon tube 200 and/or reduce a wall thickness of the balloon tube 200 prior to molding. It is contemplated that the balloon mold may have a uniform inner diameter or may have a tapered inner diameter, as desired. The balloon mold may be formed to have the desired dimensions of the balloon in the expanded configuration. For example, the balloon mold may have a body portion which tapers or reduces in the distal direction. In such an instance, both the differences in compliance of the materials of the balloon tube 200 and the distally reducing diameter of the body portion may result in a tapered balloon. Alternatively, the balloon mold may have a body portion which has a generally uniform diameter. In such an instance, as the balloon is inflated, the difference in material properties may result in uneven expansion of the balloon body portion. For example, when the proximal end region 208 is more compliant than the distal end region 210, the proximal end region 208 may radially expand to a greater extent that the distal end region 210. Thus, as the resultant balloon is expanded, the diameter of the balloon reduces in the distal direction.

The balloon tube 200 may then be adjusted to align a portion of the balloon tube with the body portion of the balloon mold, as shown at block 280. The balloon may then be molded using, for example, blow molding, as shown at block 290. During the blow molding process, the mold is heated to elevate the temperature of the polymeric material (e.g., thermoplastic material) of the balloon tube 200 in order to soften the polymeric material. While the balloon tube 200 is at an elevated temperature, the interior of the balloon tube 200 may be pressurized to expand the softened polymeric material within the cavity of the mold such that the polymeric material conforms to the shape of the cavity to form the inflatable balloon. The cavity within the balloon mold, defined by an inner surface of the balloon mold, may be configured to be the size and shape of the desired size and shape of the balloon to be formed from the balloon tube 200 within the balloon mold during the blow molding process. During a subsequent manufacturing process, the formed balloon may be bonded to a catheter shaft to form the balloon catheter (e.g., the balloon waists are bonded to the catheter shaft after the balloon is blow molded). In some examples, the balloon mold may be formed such that the resultant balloon has a consistent hoop ratio (or ID hoop) along a length of the body portion. However, this is not required. In other examples, the hoop ratio may vary along a length of the body portion of the balloon.

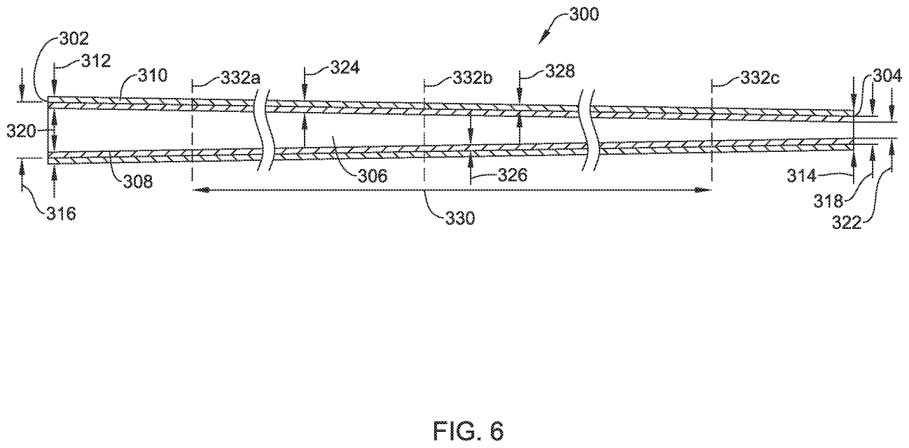

FIG. 6 is a schematic cross-section of another illustrative balloon tube 300 that may be used to form a tapered balloon, such as, but not limited to the balloon 12 described herein. The balloon tube 300 may extend from a proximal end 302 to a distal end 304. A lumen 306 may extend from the proximal end 302 to the distal end 304. Generally, the balloon tube 300 may include two or more layers, including, but not limited to, an inner layer 308 and an outer layer 310. In some examples, the inner and outer layers 308, 310 may be co-extruded as a multi-layer co-extrusion. It is contemplated that the inner layer 308 and the outer layer 310 may be formed from different materials to vary the compliance characteristics of the balloon along the length thereof. However, this is not required. The compliance characteristics may additionally or alternatively be varied by changing a wall thickness, cross-sectional dimensions, and/or material type of one or both of the layers 308, 310 along a length thereof.

In one example, the inner layer 308 may be formed from a first material and the outer layer 310 may be formed from a second material different from the first material. It is contemplated that the first material may be softer than the second material such that the inner layer 308 is more compliant than the outer layer 310. The inner layer 308 may be formed from compliant or semi-compliant materials, such as, but not limited to polyurethanes, silicone, polyether block amides (such as, but not limited to, PEBAX®), higher durometer polyurethanes, etc. The outer layer 310 may be formed from semi-compliant or non-compliant materials such as, but not limited to, polyamides, polyesters, polyether block amides (such as, but not limited to, PEBAX® or VESTAMID®). In some examples, either or both of the inner or outer layers 308, 310 may transition from a first material to a second material along a length thereof, in a manner similar to that described with respect to FIG. 4. Said differently, the material composition of the inner layer 308 and/or the outer layer 310 may vary along the length thereof.

Generally, the balloon tube 300 may be a tapered tube or a bumped tube. However, this is not required. In some examples, the balloon tube 300 may have a uniform inner and/or outer diameter. It is contemplated that one or both of the inner and outer tubes 308, 310 may reduce in inner and/or outer diameter along a length thereof. For example, the outer layer 310 of the balloon tube 300 may reduce in outer diameter from a first outer diameter 312 adjacent the proximal end 302 to a second, smaller, outer diameter 314 adjacent the distal end 304 thereof. Further, the inner layer 308 of the balloon tube 300 may reduce in outer diameter from a first outer diameter 316 adjacent the proximal end 302 to a second, smaller, outer diameter 318 adjacent the distal end 304 thereof. As the inner surface of the outer layer 310 is disposed on top of the outer surface of the inner layer, the inner diameter of the outer layer 310 may be substantially the same as the outer diameter of the inner layer 308. Thus, if the outer diameter of the inner layer 308 varies, the inner diameter of the outer layer 310 will also vary in a similar manner and if the outer diameter of the inner layer 308 remains approximately constant, the inner diameter of the outer layer 310 will remain approximately constant. In some examples, the inner layer 308 of the balloon tube 300 may reduce in outer diameter from a first outer diameter 320 adjacent the proximal end 302 to a second, smaller, outer diameter 322 adjacent the distal end 304 thereof. It is further contemplated that any of the outer diameters or inner diameters of the inner layer 308 or the outer layer 310 may remain substantially constant along a length thereof.

In the illustrated embodiment, both the outer and the inner diameters of the both the inner and outer layers 308, 310 gradually reduce towards the distal end 304. In some examples, the outer diameter of the outer layer 310 of the balloon tube 300 may have a similar slope or diameter change rate as the inner diameter of the inner layer 308 of the balloon tube 300 so that a wall thickness 324 of the balloon tube 300 remains substantially constant along a length of the balloon tube 300. In other examples, the outer diameter the outer diameter of the balloon tube 300 may have a differing slope or diameter change rate from the inner diameter of the balloon tube 300 so that a wall thickness 324 of the balloon tube 300 decreases from the proximal end 302 to the distal end 304 thereof. In some instances, the difference between the first outer diameter 312 and the second outer diameter 314 of the outer layer 310 may be greater than the difference between the first inner diameter 320 and the second inner diameter 322 of the inner layer 308. The reverse configuration in which the difference between the first inner diameter 320 and the second inner diameter 322 is greater than the difference between the first outer diameter 312 and the second outer diameter 314 is also contemplated.

It is further contemplated that a wall thickness 326 of the inner layer 308 and/or a wall thickness 328 of the outer layer 310 may be varied along a length of the balloon tube 300. For example, the ratio of the thickness 326 of the inner layer 308 to the thickness 328 of the outer layer 310 may be varied along a length of the balloon tube 300 to control the expansion profile of the balloon formed form the balloon tube 300. For example, a region of the balloon tube 300 having a higher ratio of the thickness 326 of the inner layer 308 to the thickness 328 of the outer layer 310 may expand to a greater extent that a region of the balloon tube 300 having a lower ratio of the thickness 326 of the inner layer 308 to the thickness 328 of the outer layer 310. Said differently, a thicker outer layer 310 (e.g., the less compliant material) will limit radial expansion of the balloon to a greater extent than a thinner outer layer 310. Thus, to reduce the radial growth of the balloon in the distal direction upon inflation, the wall thickness 326 of the inner layer 308 may decrease in the distal direction while the wall thickness 328 of the outer layer 310 may increase in the distal direction. In one example, the outer diameter of the outer layer 310 of the balloon tube 300 and the inner diameter of the inner layer 308 of the balloon tube 300 may remain substantially constant along a length of the balloon tube 300 and the ratio of the wall thickness 326 of the inner layer 308 to the wall thickness 328 of the outer layer 310 may be varied along a length of balloon tube 300. In another example, the outer diameter of the outer layer 310, the outer diameter of the inner layer 308, and/or the inner diameter of the inner layer 308 may reduce in the distal direction along with varying a ratio of the thickness 326 of the inner layer 308 to the thickness 328 of the outer layer 310.

During the blow molding process, an intermediate portion 330 of the balloon tube 300 may form the body portion of the balloon. The intermediate portion 330 may extend from a proximal diameter transition point 332a to a distal diameter point 332c. To form a balloon having a body portion length of about 300 mm, the intermediate portion 330 may have a length in the range of about 4.6 inches (116.8 mm) to about 4.8 inches (121.9 mm), or about 4.7 inches (119.4 mm). However, the length of the intermediate portion 330 may be selected to form the desired balloon length. For example, a shorter balloon body portion may be formed from a shorter balloon tube and a longer balloon body portion may be formed from a longer balloon tube. At the proximal diameter transition point 332a, the outer diameter of the outer layer 310 may be in the range of about 0.039 inches (0.99 mm) to about 0.043 inches (1.09 mm), or about 0.041 inches (1.04 mm), an outer diameter of the inner layer 308 may be in the range of about 0.031 inches (0.79 mm) to about 0.035 inches (0.89 mm), or about 0.033 inches (0.84 mm), and the inner diameter of the inner layer 308 may be in the range of about 0.023 inches (0.58 mm) to about 0.027 inches (0.69 mm), or about 0.025 inches (0.64 mm). At the distal diameter transition point 332c, the outer diameter of the outer layer 310 may be in the range of about 0.031 inches (0.79 mm) to about 0.035 inches (0.89 mm), or about 0.033 inches (0.84 mm), the outer diameter of the inner layer 308 may be in the range of about 0.025 inches (0.64 mm) to about 0.029 inches (0.74 mm), or about 0.027 inches (0.69 mm), and the inner diameter may be in the range of about 0.018 inches (0.46 mm) to about 0.022 inches (0.56 mm) or about 0.020 inches (0.51 mm). An intermediate outer diameter of the outer layer 310, for example, at or near the second diameter transition point 332b, may be in the range of about 0.035 inches (0.89 mm) to about 0.039 inches (0.99 mm) or about 0.037 inches (0.94 mm), an intermediate outer diameter of the inner layer 308 may be in the range of about 0.028 inches (0.71 mm) to about 0.032 inches (0.81 mm), or about 0.030 inches (0.76 mm), and an intermediate inner diameter of the inner layer 308 may be in the range of about 0.020 inches (0.51 mm) to about 0.024 inches (0.61 mm) or about 0.022 inches (0.56 mm). It is contemplated that the outer diameters, inners diameters, and/or a rate of change of the outer and/or inner diameters may be varied, as desired.

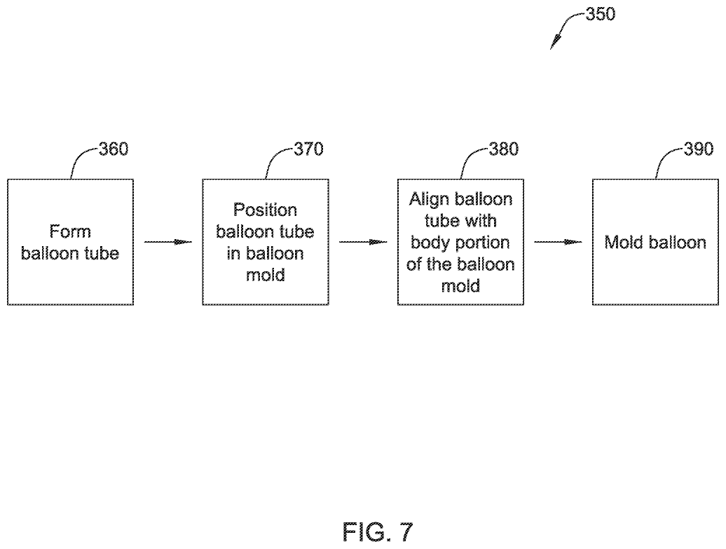

FIG. 7 is an illustrative flowchart of a method 350 for forming a tapered balloon using a multi-layer tube, such as, but not limited to, balloon tube 300. To begin, the balloon tube 300 may be formed, as shown at block 360. In some embodiments, the balloon tube 300 may be co-extruded as a single monolithic structure including two or more layers. In other embodiments, the balloon tube 300 may be formed as separate components that are subsequently coupled or bonded. As described above, the inner and/or outer diameters of the inner layer 308 and/or the outer layer 310 may be varied along a length of the balloon tube 300. Additionally, or alternatively, the materials of the inner layer 308 and/or the outer layer 310 may be varied and/or a wall thickness of the inner layer 308 and/or outer layer 310 may be varied. The balloon tube 300 may then be placed into a balloon mold, as shown at block 370. In some examples, the balloon tube 300 may be pre-stretched prior to positioning the balloon tube 300 into the tapered balloon mold to increase a length of the balloon tube 300 and/or reduce a wall thickness of the balloon tube 300 prior to molding. It is contemplated that the balloon mold may have a uniform inner diameter or may have a tapered inner diameter, as desired. The balloon mold may be formed to have the desired dimensions of the balloon in the expanded configuration. For example, the balloon mold may have a body portion which tapers or reduces in the distal direction. In such an instance, the differences in compliance of the materials of the balloon tube 300, the thickness of the inner and/or outer layers 308, 310, and the distally reducing diameter of the body portion may result in a tapered balloon. Alternatively, the balloon mold may have a body portion which has a generally uniform diameter. In such an instance, as the balloon is inflated, the difference in material properties and/or the thickness of the inner and/or outer layers 308, 310 may result in uneven expansion of the balloon body portion. For example, the material properties and/or the thickness of the inner and/or outer layers 308, 310 may be varied such that, the proximal end 302 may radially expand to a greater extent that the distal end 304. Thus, as the resultant balloon is expanded, the diameter of the balloon reduces in the distal direction.

The balloon tube 300 may then be adjusted to align a portion of the balloon tube with the body portion of the balloon mold, as shown at block 380. In some examples, this may include aligning a proximal diameter transition point (e.g., transition point 332a in FIG. 6) with the proximal end of the body portion of the mold and a distal diameter transition point (e.g., transition point 332c in FIG. 6) with the distal end of the body portion of the mold. The balloon may then be molded using, for example, blow molding, as shown at block 390. During the blow molding process, the mold is heated to elevate the temperature of the polymeric material (e.g., thermoplastic material) of the balloon tube 300 in order to soften the polymeric material. While the balloon tube 300 is at an elevated temperature, the interior of the balloon tube 300 may be pressurized to expand the softened polymeric material within the cavity of the mold such that the polymeric material conforms to the shape of the cavity to form the inflatable balloon. The cavity within the balloon mold, defined by an inner surface of the balloon mold, may be configured to be the size and shape of the desired size and shape of the balloon to be formed from the balloon tube 300 within the balloon mold during the blow molding process. During a subsequent manufacturing process, the formed balloon may be bonded to a catheter shaft to form the balloon catheter (e.g., the balloon waists are bonded to the catheter shaft after the balloon is blow molded). In some examples, the balloon mold may be formed such that the resultant balloon has a consistent hoop ratio (or ID hoop) along a length of the body portion. However, this is not required. In other examples, the hoop ratio may vary along a length of the body portion of the balloon.

It is contemplated that the balloons formed from the balloon tubes 100, 200, 300 may include one or more cutting blades or scoring members affixed to an outer surface thereof. Some illustrative cutting blades or scoring members are described in commonly assigned U.S. Pat. No. 8,038,691, titled CUTTING BALLOON CATHETER HAVING FLEXIBLE ATHEROTOMES, U.S. Pat. No. 7,153,315, titled CATHETER BALLOON WITH ULTRASONIC MICROSCALPEL BLADES, and U.S. Patent Publication Number 2021/0153891, titled CUTTING BALLOON CATHETER, the disclosures of which are hereby incorporated by reference. When so provided, the cutting blades and/or scoring members may be spaced about a length and/or circumference of the balloons, as desired.

It is contemplated that the balloon tubes 100, 200, 300 and/or the balloons formed therefrom may be coated with a therapeutic agent. Some examples of suitable therapeutic agents may include anti-thrombogenic agents (such as heparin, heparin derivatives, urokinase, and PPack (dextrophenylalanine proline arginine chloromethylketone)); antiproliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

The materials that can be used for the various components of the system(s) and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the catheter shaft, the inflatable balloon, the expandable basket, the central wire, etc., and/or elements or components thereof.

In some embodiments, the system, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), expanded polytetrafluoroethylene (ePTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments components can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system, and/or components or portions thereof, may be made of a material that does not substantially distort the image and create substantial artifacts (i.e., gaps in the image). Certain ferromagnetic materials, for example, may not be suitable because they may create artifacts in an MRI image. The system, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the present disclosure. This may include, to the extent that it is appropriate, the use of any of the features of one example embodiment being used in other embodiments. The scope of the present disclosure is, of course, defined in the language in which the appended claims are expressed.

What is claimed is:

1. A method for forming a tapered balloon for use in a medical device, the method comprising:
forming a tapered balloon tube, the tapered balloon tube decreasing in outer diameter from a proximal end to a distal end;
positioning the tapered balloon tube within a balloon mold, the balloon mold having a body portion that reduces in diameter in a distal direction; and
blow molding the tapered balloon tube within the balloon mold to form a balloon.

2. The method of claim 1, wherein positioning the tapered balloon tube within the balloon mold comprises aligning a proximal diameter transition point of the balloon tube with a proximal end of the body portion of the balloon mold.

3. The method of claim 1, wherein a hoop ratio of the balloon mold and the balloon tube is consistent along a length of a body portion of the formed balloon.

4. The method of claim 1, wherein the tapered balloon tube is formed from a single material.

5. A method for forming a tapered balloon for use in a medical device, the method comprising:
forming a balloon tube, the balloon tube formed from a first material and a second material different from the first material;
positioning the balloon tube within a balloon mold; and
blow molding the tapered balloon tube within the balloon mold to form a balloon.

6. The method of claim 5, wherein the first material is more compliant than the second material.

7. The method of claim 5, wherein the balloon tube decreases in outer diameter from a proximal end to a distal end.

8. The method of claim 5, wherein an outer diameter of the balloon tube is uniform from a proximal end to a distal end.

9. The method of claim 5, wherein the balloon tube comprises a proximal end region comprising the first material and a distal end region comprising the second material.

10. The method of claim 9, wherein the proximal end region is more compliant than the distal end region.

11. The method of claim 9, wherein the balloon tube further comprises a transition region between the proximal end region and the distal end region.

12. The method of claim 11, wherein the transition region comprises a gradient transition from the first material to the second material.

13. The method of claim 5, wherein the balloon tube comprises an inner layer comprising the first material and an outer layer comprising the second material.

14. The method of claim 13, wherein a wall thickness of the inner layer decreases from a proximal end to a distal end of the balloon tube.

15. The method of claim 13, wherein a wall thickness of the outer layer increases from a proximal end to a distal end of the balloon tube.

16. A method for forming a tapered balloon for use in a medical device, the method comprising:
forming a tapered balloon tube, the tapered balloon tube decreasing in outer diameter from a proximal end to a distal end and formed from a first material and a second material, wherein the first material is more compliant that the second material;
positioning the balloon tube within a balloon mold; and
blow molding the tapered balloon tube within the balloon mold to form a balloon.

17. The method of claim 16, wherein the balloon tube comprises a proximal end region comprising the first material and a distal end region comprising the second material.

18. The method of claim 17, wherein the balloon tube further comprises a transition region between the proximal end region and the distal end region.

19. The method of claim 16, wherein the balloon tube comprises an inner layer comprising the first material and an outer layer comprising the second material.

20. The method of claim 19, wherein a ratio of a wall thickness of the inner layer to a wall thickness of the outer layer is varied along a length of the balloon tube.
